(19) United States
(12) Patent Application Publication
(10) Pub. No.: US 2021/0275075 A1
(43) Pub. Date: Sep. 9, 2021

(54) BIOELECTRODE AND METHOD OF MANUFACTURING THE SAME

(71) Applicant: Panasonic Intellectual Property Management Co., Ltd., Osaka (JP)

(72) Inventor: KEI TOYOTA, Osaka (JP)

(21) Appl. No.: 17/180,916

(22) Filed: Feb. 22, 2021

(30) Foreign Application Priority Data
Mar. 4, 2020 (JP) 2020-037169

(51) Int. Cl. A61B 5/25; C08G 65/08; C08G 77/32; C01B 32/158; C01B 32/20

(52) U.S. Cl. CPC A61B 5/25; C08G 65/08; C08G 77/32; C01B 32/158; B82Y 5/00; A61B 2562/125; C01P 2006/40; C01P 2004/13; C01P 2004/64; C01B 32/20

(57) ABSTRACT

A bioelectrode includes an inorganic base material and a conductive layer covering the inorganic base material, in which the conductive layer has a polymer having moieties derived from a first compound having an epoxy group and an alkoxysilyl group, and at least one of an alkali metal ion and a Group 2 element ion supported in the polymer, and in the polymer, the moiety derived from the epoxy group is ring-opening polymerized, and the moiety derived from the alkoxysilyl group forms a siloxane bond.

### BACKGROUND

### 1. Technical Field

The present disclosure relates to a bioelectrode and a method of manufacturing the same.

### 2. Description of the Related Art

A bioelectrode is mainly required to detect a potential signal transmitted through a nervous system of a human body, and is used for electrocardiogram and electroencephalography at a position close to the human body.

From now on, in order to realize a society in which the human body and machines are integrated so that machines and robots can be operated at will, there is a demand for measuring, analyzing, and converting a potential signal with high accuracy to transmit the potential signal to an electrical device or the like. Therefore, a bioelectrode capable of detecting a potential signal from a living body with higher accuracy is required.

Japanese Patent Unexamined Publication No. 2015-41419 discloses a bioelectrode having carbon material powders such as carbon nanotubes mixed with rubber, and International Publication No. 2019/139165 discloses a bioelectrode having a silicone rubber sheet in which silver particles are mixed.

### SUMMARY

According to an aspect of the present disclosure, a bioelectrode includes an inorganic base material and a conductive layer covering the inorganic base material, in which the conductive layer has a polymer having moieties derived from a first compound having an epoxy group and an alkoxysilyl group, and at least one of an alkali metal ion and a Group 2 element ion supported in the polymer, and in the polymer, the moiety derived from the epoxy group is ring-opening polymerized, and the moiety derived from the alkoxysilyl group forms a siloxane bond.

According to an aspect of the present disclosure, a method of manufacturing a bioelectrode includes preparing a solution in which at least one of an alkali metal salt and a Group 2 element salt is dissolved in a liquid including a first compound having an epoxy group and an alkoxysilyl group, applying the solution to an inorganic base material, and curing the applied solution.

### BRIEF DESCRIPTION OF THE DRAWINGS

FIG. 1 is a conceptual diagram for explaining a structure of a bioelectrode according to an exemplary embodiment of the present disclosure in a case where γ-glycidoxypropylmethyldimethoxysilane is used as a first compound and lithium ions (Lit) are used as alkali metal ions;

FIG. 2A shows a total reflection FTIR spectrum of a solution in Example 1;

FIG. 2B shows a total reflection FTIR spectrum of a conductive layer in Example 1;

FIG. 3 shows an ultraviolet visible absorption spectrum of the conductive layer in Example 1;

FIG. 4 shows a GPC measurement result in Example 5;

FIG. 5 shows an ultraviolet visible absorption spectrum of the conductive layer in Example 5; and

FIG. 6 is a table summarizing evaluation results in Examples 1 to 13 and Comparative Examples 1 to 3.

### DETAILED DESCRIPTIONS

Bioelectrodes of Japanese Patent Unexamined Publication No. 2015-41419 and International Publication No. 2019/139165 are intended to measure potential change from a surface of a human body (skin), and are flexible because they are made of rubber. Such electrodes that easily change a shape are not suitable for obtaining signals from a deep part of the human body or a fine part such as a nerve cell with high accuracy. On the other hand, if an electrode made of a hard metal is simply used as a bioelectrode for a long period of time, measurement accuracy is reduced due to electrolysis or the like, and in some cases, the metal may cause allergic symptoms to the human body.

An exemplary embodiment of the present disclosure has been made in view of such a situation, an object thereof is to provide a bioelectrode that has a high rigidity and whose surface is mainly made of a non-metallic material to come into contact with a living body.

The inventors of the present application have studied from various angles to realize a bioelectrode that has high rigidity and whose surface is mainly made of a non-metallic material to come into contact with a living body.

As a result, the inventors of the present application have found a bioelectrode and a manufacturing method thereof, the bioelectrode including an inorganic base material and a conductive layer covering the inorganic base material, in which the conductive layer includes a polymer having moieties derived from a first compound having an epoxy group and an alkoxysilyl group, and at least one of an alkali metal ion and a Group 2 element ion supported in the polymer.

According to Aspect 1 of the present disclosure, a bioelectrode includes an inorganic base material and a conductive layer covering the inorganic base material, in which the conductive layer has a polymer having moieties derived from a first compound having an epoxy group and an alkoxysilyl group, and at least one of an alkali metal ion and a Group 2 element ion supported in the polymer, and in the polymer, the moiety derived from the epoxy group is ring-opening polymerized, and the moiety derived from the alkoxysilyl group forms a siloxane bond.

Aspect 2 of the present disclosure is the bioelectrode according to Aspect 1 in which the epoxy group constitutes a glycidyl ether group.

Aspect 3 of the present disclosure is the bioelectrode according to Aspect 1 or Aspect 2 in which the siloxane bond is formed by the moiety derived from the alkoxysilyl group of the first compound, and a moiety derived from an alkoxysilyl group of a second compound having a hydrocarbon group and the alkoxysilyl group.

Aspect 4 of the present disclosure is the bioelectrode according to any one of Aspects 1 to 3, in which the inorganic base material is in a fibrous form having a circle equivalent diameter of 100 μm or more and 5 mm or less.

Aspect 5 of the present disclosure is the bioelectrode according to Aspect 4 in which the inorganic base material has a pointed structure portion.

Aspect 6 of the present disclosure is the bioelectrode according to Aspect 4 or 5, in which the inorganic base material includes glass or a metal.

Aspect 7 of the present disclosure is the bioelectrode according to any one of Aspects 1 to 6, in which the polymer further has a cyclic polyether structure.

Aspect 8 of the present disclosure is the bioelectrode according to any one of Aspects 1 to 7, in which the conductive layer further includes conductive particles.

Aspect 9 of the present disclosure is the bioelectrode according to Aspect 8, in which the conductive particles include at least one or more selected from the group consisting of carbon, silver, and copper, and have an average particle diameter of 0.5 nm or more and 100 μm or less.

Aspect 10 of the present disclosure is the bioelectrode according to Aspect 8 or 9, in which the conductive particles are at least one of carbon nanotubes and graphite powders.

Aspect 11 of the present disclosure is a method of manufacturing a bioelectrode including preparing a solution in which at least one of an alkali metal salt and a Group 2 element salt is dissolved in a liquid including a first compound having an epoxy group and an alkoxysilyl group, applying the solution to an inorganic base material, and curing the applied solution.

Aspect 12 of the present disclosure is the manufacturing method according to Aspect 11 further including mixing conductive particles with the solution after the preparing of the solution and before the applying of the solution to the inorganic base material.

Aspect 13 of the present disclosure is the manufacturing method according to Aspect 11 or 12, in which the liquid further includes a second compound having a hydrocarbon group and an alkoxysilyl group.

According to the above aspects of the present disclosure, it is possible to provide a bioelectrode that has high rigidity and whose surface is mainly made of a non-metallic material to come into contact with a living body.

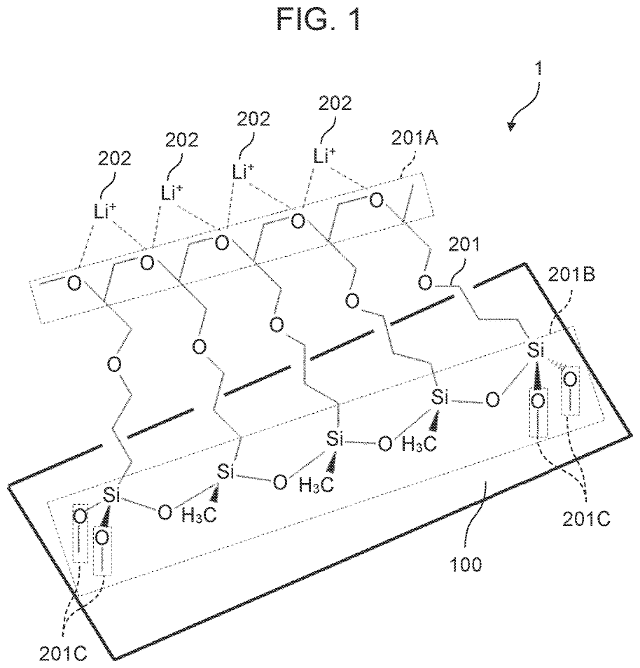

FIG. 1 is a conceptual diagram for explaining a structure of a bioelectrode according to an exemplary embodiment of the present disclosure in a case where γ-glycidoxypropylmethyldimethoxysilane is used as a first compound and lithium ions (Lit) are used as alkali metal ions.

As illustrated in FIG. 1, bioelectrode 1 according to the exemplary embodiment of the present disclosure includes a conductive layer covering inorganic base material 100. The conductive layer contains polymer 201 containing moieties derived from a first compound, and polymer 201 supports at least one of an alkali metal ion and Group 2 element ion 202 (in FIG. 1, lithium ion (Lit)). Polymer 201 has portion 201A that is subjected to ring-opening polymerization of a moiety derived from an epoxy group (hereinafter, referred to as “polyethylene oxide structure 201A”) and portion 201B that is subjected to hydrolyzation and condensation of a moiety derived from an alkoxysilyl group to form a siloxane bond (hereinafter, referred to as “siloxane bond structure 201B”). Siloxane bond structure 201B may have portions 201C that are subjected to bonding of the conductive layer to inorganic base material 100 (hereinafter, referred to as “base material/conductive layer bonding portions 201C”).

In the structure illustrated in FIG. 1, the following (1) to (4) are shown: (1) rigidity of inorganic base material 100 can be improved, (2) the conductive layer, which is a surface of the bioelectrode for coming into contact with a living body, is mainly made of a non-metallic material, (3) adhesion between the conductive layer and inorganic base material 100 can be secured by siloxane bond structure 201B, and (4) ion conductivity can be secured by at least one of polyethylene oxide structure 201A, the alkali metal ion, and Group 2 element ion 202.

Hereinafter, the bioelectrode according to the exemplary embodiment of the present disclosure will be described.

Inorganic Base Material

Inorganic base material 100 according to the exemplary embodiment of the present disclosure is made of a metal, glass or ceramics, or a mixture thereof, as a main component (that is, a content of the main component exceeds 50% by mass with respect to the total mass of the base material). By using such an inorganic base material, it is possible to obtain a bioelectrode having high rigidity.

Inorganic base material 100 preferably contains an inorganic oxide. A hydroxyl group may be formed on a surface of the base material containing an inorganic oxide. The hydroxyl group can be subjected to a dehydration condensation reaction with the hydrolyzed alkoxysilyl group of the first compound (that is, a silanol group) to form base material/conductive layer bonding portions 201C. Therefore, the adhesion of inorganic base material 100 containing an inorganic oxide with the conductive layer can be improved.

Examples of the inorganic oxide suitably used for inorganic base material 100 can include glass. Examples of the glass can include borosilicate glass using silicon dioxide as a main raw material, BK7, synthetic quartz, anhydrous synthetic quartz, soda-lime glass, and crystalline glass. Further, these glasses may contain alumina, calcium oxide, magnesium oxide, boric acid, sodium oxide, potassium oxide, and the like.

Moreover, inorganic base material 100 preferably contains a metal. This is because in the base material containing a metal, an oxide can be formed thinly on a surface of the metal, and a hydroxyl group can be formed on the outermost surface thereof.

Examples of the metal suitably used for inorganic base material 100 can include platinum, gold, silver, copper, stainless steel, and aluminum.

A shape of inorganic base material 100 is not particularly limited. However, preferably, inorganic base material 100 is in a fibrous form having a circle equivalent diameter of 100 μm or more and 5 mm or less in a cross-sectional direction. With such a shape, a contact area with a deep part of a human body or a fine part such as a nerve cell can be reduced, and an electric signal from the fine part can be measured with higher accuracy. On the other hand, the shape of inorganic base material 100 may be formed into a plate shape on the assumption that inorganic base material 100 is attached to the surface of the human body to measure the electric signal.

The term “fibrous form” as used herein refers to an elongated shape like fiber, which has a cross-sectional direction and a length direction and has a length in the length direction longer than the maximum length in the cross-sectional direction. When inorganic base material 100 is in a fibrous form, a cross-sectional shape thereof is not particularly limited. However, examples of the cross-sectional shape can include a circle, an ellipse, a square, and a triangle. When inorganic base material 100 has different cross-sectional shapes in the length direction, the smaller circle equivalent diameter among the circle equivalent diameters in the cross-sectional direction from both ends of the length direction may be 100 μm or more and 5 mm or less. Examples of the cross-sectional shape of the fibrous form can include a columnar form, a prismatic form, a fibrous form, a needle-like form, and a conical form. In a case where the cross-sectional shape is a columnar form or a fibrous form (that is, in a case where the cross-sectional shape is circular), an outer diameter of the inorganic base material is preferably 100 μm or more and 5 mm or less.

In order to measure the electric signal from the fine part with higher accuracy, it is more preferable that inorganic base material 100 has a pointed structure portion. As a result, the contact area with the deep part of the human body or the fine part such as a nerve cell can be made smaller.

### Conductive Layer

The conductive layer according to the exemplary embodiment of the present disclosure contains polymer 201 having moieties derived from the first compound having an epoxy group and an alkoxysilyl group, and at least one of an alkali metal ion and Group 2 element ion 202 supported in polymer 201. In polymer 201, the moiety derived from the epoxy group is ring-opening polymerized to form polyethylene oxide structure 201A, and the moiety derived from the alkoxysilyl group forms siloxane bond structure 201B. Further, siloxane bond structure 201B may have base material/conductive layer bonding portions 201C. That is, the moiety derived from the alkoxysilyl group may form a siloxane bond and bond the siloxane bond with the surface of the inorganic base material.

Further, in the conductive layer, a polymer chain grows three-dimensionally in addition to the above-described bonding structure with the inorganic base material. Specifically, the moiety that is subjected to ring-opening polymerization of the moiety derived from the epoxy group forms a polymer chain to be away from the surface of the inorganic base material, and the moiety derived from the alkoxysilyl group forms a siloxane bond at a position away from the surface of the inorganic base material. The thickness of the conductive layer is not particularly limited, but can be adjusted as appropriate.

The first compound according to the exemplary embodiment of the present disclosure has an epoxy group and an alkoxysilyl group, and can be represented by the following General Formula (1).

GCₙH_{2n-2m-4f}SiR²_{3-g}(OR³)_{g} (1)

In General Formula (1), G may be a functional group having an epoxy group, and examples of the functional group having an epoxy group can include a glycidyl ether group and an epoxycyclohexyl group, but a glycidyl ether group is preferably used from the viewpoint of high reactivity and easiness to obtain a polymer. That is, in the exemplary embodiment of the present disclosure, it is preferable that the epoxy group constitutes a glycidyl ether group.

R² and R³ may be, independently for each occurrence, any of a methyl group, an ethyl group, a propyl group, a butyl group, an isopropyl group, a pentyl group, an isobutyl group, a hexyl group, a phenyl group, and a cyclohexyl group, and R² and R³ may be the same or different. A methyl group and an ethyl group can be preferably used from the viewpoint of easiness of hydrolyzation and high adsorptivity on the surface of inorganic base material 100, particularly, a surface of a glass base material.

n may be an integer of 0 or more and 8 or less. By setting n to 8 or less, hydrophobicity of the first compound can be suppressed from being excessively increased, and solubility of at least one of the alkali metal salt and the Group 2 element salt in the liquid of the first compound can be secured. Further, when epoxy groups are ring-opening polymerized, a distance between silicon (Si) atoms is secured, such that n is preferably 3 or more from the viewpoint that steric hindrance due to an alkoxy group (OR³) bonded to the Si atom can be suppressed. In hydrocarbon represented by CₙH_{2n-2m-4f}, m is a total of the number of double bonds and the number of cyclic structures in the hydrocarbon, and f is the number of triple bonds in the hydrocarbon.

g is an integer of 1 or more and 3 or less. As g becomes smaller, a ratio of bonding between alkoxy groups is reduced, and volume shrinkage during polymerization can be suppressed, such that generation of internal cracks in the conductive layer due to the volume shrinkage of the polymer can be suppressed. On the other hand, as g becomes larger, the number of alkoxy groups that contributes to the formation of base material/conductive layer bonding portion 201C increases, and therefore, the adhesion between the conductive layer and inorganic base material 100 is improved. From the viewpoint of achieving the suppression of internal cracks and the adhesion, g is preferably 2.

Examples of the first compound can include γ-glycidoxypropyltrimethoxysilane, γ-glycidoxypropylmethyldimethoxysilane, γ-glycidoxypropyltriethoxysilane, γ-glycidoxypropylmethyldiethoxysilane, 2-(3,4-epoxycyclohexyl)trimethoxysilane, 2-(3,4-epoxycyclohexyl)methyldimethoxysilane, 2-(3,4-epoxycyclohexyl)triethoxysilane, and 2-(3,4-epoxycyclohexyl)methyldiethoxysilane.

Polymer 201 containing the moiety derived from the first compound according to the exemplary embodiment of the present disclosure supports at least one of the alkali metal ion and Group 2 element ion 202, the moiety derived from the epoxy group is ring-opening polymerized, and the moiety derived from the alkoxysilyl group forms a siloxane bond.

When the moiety derived from the epoxy group of the first compound is ring-opening polymerized by, for example, at least one of an alkali metal salt and a Group 2 element salt, polyethylene oxide structure 201A may be formed as a main structure. When polyethylene oxide structure 201A is formed to support at least one of the alkali metal ion and Group 2 element ion 202, the conductive layer containing polymer 201 has ion conductivity.

It is preferable that in the conductive layer, the epoxy group of the first compound is cyclically polymerized to form a polymer having a cyclic polyether structure. As a result, at least one of the alkali metal ion and Group 2 element ion 202 can be stably supported, and the ion conductivity can thus be improved. From the viewpoint that alkali metal ions such as lithium ion, sodium ion, and potassium ion can be easily supported, it is preferable that four or more and six or less epoxy groups are cyclically polymerized.

Since the polymer having a cyclic polyether structure absorbs ultraviolet rays, a content of the polymer is defined with an ultraviolet part, for example, an absorbance at 450 nm in an ultraviolet visible absorption spectrum. Here, the absorbance of the conductive layer at 450 nm is preferably 0.200 or more. By keeping this range, a sufficient content of the polymer having a cyclic polyether structure can be secured, and the ion conductivity is further improved. On the other hand, since it takes time to form the cyclic polyether structure, the absorbance of the conductive layer at 450 nm is preferably 2.50 or less from the viewpoint of productivity.

Examples of the alkali metal ion supported in polymer 201 can include lithium ion, sodium ion, and potassium ion. Examples of the Group 2 element ion supported in polymer 201 can include magnesium ion, calcium ion, and strontium ion.

The alkoxysilyl group of the first compound is mainly hydrolyzed to form a silanol group, and the silanol groups can be dehydrated and condensed to form siloxane bond structure 201B. Since the conductive layer having siloxane bond structure 201B may have base material/conductive layer bonding portions 201C, the conductive layer is suitable for securing the adhesion with the inorganic base material.

Siloxane bond structure 201B may be formed by the moiety derived from the alkoxysilyl group of the first compound and a moiety derived from an alkoxysilyl group of a second compound having a hydrocarbon group and the alkoxysilyl group. In a case where siloxane bond structure 201B is formed by the first compound and the second compound, a cross-link density can be reduced to suppress the occurrence of cracks in the conductive layer, as compared to a case where siloxane bond structure 201B is formed by only the first compound. Further, when the cross-link density is reduced, more ion conduction paths are formed and the ion conductivity of bioelectrode 1 is improved, which is preferable.

The second compound can be represented by the following General Formula (2).

XₚY_{q}ZᵣSi(OR⁴)ₐ(OR⁵)_{b}(OR⁶)_{c} (2)

X, Y, and Z are not particularly limited, but can be, for example, a hydrocarbon group represented by general formula of CₛH₂ₛ₊₁₋₂ₜ₋₄ᵤ. s can be 1 or more and 20 or less. By setting s to 20 or less, it is possible to prevent excessively serious steric hindrance and relatively easily form a polymer. t is a total of the number of double bonds and the number of cyclic structures in the hydrocarbon group, and u is the number of triple bonds in the hydrocarbon group. All the X, Y, and Z may be the same or different.

Specific examples of X, Y, and Z can include a methyl group, an ethyl group, a propyl group, a butyl group, a hexyl group, a phenyl group, a cyclohexyl group, an octyl group, a decyl group, and an allyl group.

R⁴, R⁵, and R⁶ may be hydrocarbon groups, and preferably alkyl groups having 1 or more and 5 or less carbon atoms.

p, q, r, a, b, and c are integers of 0 or more that satisfy 1≤p+q+r≤3, 1≤a+b+c≤3, and p+q+r+a+b+c=4.

The second compound may be a mixture of compounds represented by Formula (2) described above. When the second compound is a mixture, the second compound is a mixture of a compound containing two alkoxysilyl groups (that is, a+b+c) and a compound containing three alkoxysilyl groups. Therefore, the cross-link density of siloxane bond structure 201B can be adjusted to obtain a conductive layer that achieves the suppression of internal cracks and the adhesion.

As an amount of the second compound added, a ratio of the number of moles of the second compound to the total of the number of moles of the first compound and the number of moles of the second compound can be 0.1 or more and 0.5 or less. When the ratio of the number of moles of the second compound to the total of the number of moles of the first compound and the number of moles of the second compound is 0.1 or more, an effect of reducing the cross-link density is easily obtained. When the ratio of the number of moles of the second compound to the total of the number of moles of the first compound and the number of moles of the second compound is 0.5 or more, a density of polyethylene oxide structure 201A can be increased, and sufficient conductivity is thus easily obtained.

The conductive layer may contain conductive particles. As a result, an electrical resistance of the bioelectrode can be reduced. Examples of the conductive particles can include, but are not limited to, carbon materials such as graphite powders and carbon nanotubes, and metal materials such as silver and copper. In the conductive layer, the conductive particles are covered with polymer 201.

An average particle diameter of the conductive particle is 0.5 nm or more, which is preferable. By setting the average particle diameter to 0.5 nm or more, it is possible to suppress the aggregation of the particles, thereby dispersing the conductive particles more uniformly. Even if the conductive particles are too large, it is difficult to uniformly disperse the conductive particles in the conductive layer due to the influence on sedimentation or the like, such that the average particle diameter of the conductive particle is preferably 100 μm or less. Furthermore, when the average particle diameter of the conductive particle is 100 nm or more and 50 μm or less, it is easy to achieve dispersibility and conductivity, which is more preferable.

The “average particle diameter” as used herein means a volume standard median size (D50).

An amount of the conductive particles added in the conductive layer is preferably 10% or more in terms of a volume ratio. When the volume ratio is 10% or more, a distance between the particles can be kept within a certain distance, and the conductivity can be improved. More preferably, the volume ratio is 30% or more. On the other hand, in order to suppress air inclusion and exposure to the surface to obtain a uniform polymer, the amount of the conductive particles added is preferably 60% or less in terms of a volume ratio. More preferably, the amount of the conductive particles added is 50% or less.

A shape of the conductive particle may be, but not limited to, a fibrous form in addition to a particulate form. When the shape of the conductive particle is a fibrous form, adjacent particles are likely to come into contact with each other, which is preferable in improvement of ion conductivity.

As long as the object of the exemplary embodiment of the present disclosure is achieved, the bioelectrode according to the exemplary embodiment of the present disclosure may contain other components.

Hereinafter, a method of manufacturing a bioelectrode according to the exemplary embodiment of the present disclosure will be described.

The method of manufacturing a bioelectrode according to the exemplary embodiment of the present disclosure includes:
(a) Process of preparing a solution,
(b) Process of applying the solution to an inorganic base material, and
(c) Process of curing the applied solution.

### (a) Solution Preparing Process

A solution in which at least one of an alkali metal salt and a Group 2 element salt is dissolved in a liquid containing a first compound having an epoxy group and an alkoxysilyl group is prepared. The dissolution method is not particularly limited, but it is preferable that the solution is heated and stirred at a temperature of 30° C. or higher and 60° C. or lower for 1 minute or longer. When the solution is heated and stirred at 30° C. or higher, it can be easily dissolved. When the solution is heated and stirred at 60° C. or lower, it is possible to suppress the polymerization in a solution preparation stage and increased viscosity of the solution due to the polymerization, and suppress thickness variation when applying the solution to the inorganic base material. A heating and stirring time is preferably 10 minutes or longer. When stirring, for example, a stirrer or the like can be used.

In Process (a), at least one of the alkali metal salt and the Group 2 element salt is not particularly limited, but the solution contains a combination of cation of at least one of an alkali metal and Group 2 element and anion of at least one of the corresponding alkali metal and Group 2 element. Examples of the cation can include lithium ion, sodium ion, potassium ion, magnesium ion, calcium ion, and strontium ion. Examples of the anion can include chloride ion, bromide ion, iodide ion, perchlorate ion, thiocyanate ion, tetrafluoroborate ion, nitrate ion, sulfate ion, hexafluoroarsenic acid ion (AsF₆⁻), and hexafluorophosphoric acid ion (PF₆⁻). As at least one of the alkali metal salt and the Group 2 element salt, lithium perchlorate and sodium iodide are preferable from the viewpoint of high solubility in the first compound having an alkoxysilyl group.

In Process (a), an amount of at least one of the alkali metal salt and Group 2 element salt added is preferably the number of moles of 5% or more and 30% or less with respect to the number of moles of the first compound. When the amount of at least one of the alkali metal salt and Group 2 element salt added is 5% or more, the polyethylene oxide structure can be sufficiently formed. When the amount of at least one of the alkali metal salt and Group 2 element salt added is 30% or less, it is possible to suppress production of a precipitate of the salt in the solution.

In Process (a), the liquid may contain a second compound containing a hydrocarbon group and a hydrolyzable silyl group. As a result, siloxane bond structure 201B can be formed by the moiety derived from the alkoxysilyl group of the first compound and a moiety derived from an alkoxysilyl group of a second compound having a hydrocarbon group and the alkoxysilyl group.

After Process (a) and before Process (b), a process of mixing the conductive particles with the solution may be further included. As a result, a conductive layer can further contain the conductive particles. The mixing method is not particularly limited, but for example, the conductive particles may be added to the solution and then shaken manually, or may be stirred with a stirrer or the like.

### (b) Applying Process

The solution obtained in Process (a) is applied to an inorganic base material. The applying method is not particularly limited, but for example, the applying may be carried out by immersing the inorganic base material in the solution. If the inorganic base material has a plate shape, it may be applied with a spin coater or the like.

### (c) Curing Process

The solution applied in Process (b) is cured. In this process, ring-opening polymerization of an epoxy group by metal cation of at least one of the alkali metal salt and the Group 2 element salt can proceed to form polyethylene oxide structure 201A. In addition, at least one of the alkali metal ion and Group 2 element ion 202 may be supported by the coordination bond from an oxygen atom contained in polyethylene oxide structure 201A.

In addition, the hydrolysis of the alkoxysilyl group can proceed due to moisture in the atmosphere to produce a silanol group at the same time. Further, the produced silanol groups are dehydrated and condensed to form siloxane bond structure 201B, the solution is cured, and polymer 201 of the first compound (and the second compound) and a conductive layer containing polymer 201 are thus formed. The produced silanol group can be subjected to a dehydration condensation reaction with the hydroxyl group on the surface of inorganic base material 100 to form base material/conductive layer bonding portions 201C. That is, the moiety derived from the alkoxysilyl group may form a siloxane bond and bond the siloxane bond with the surface of the inorganic base material. As a result, the conductive layer and inorganic base material 100 are well bonded to each other to obtain bioelectrode 1 having excellent adhesion.

A curing time in Process (c) is preferably 20 minutes or longer, and more preferably, 30 minutes or longer, 1 hour or longer, 24 hours or longer, 100 hours or longer, 500 hours or longer, or 720 hours or longer. As a result, the polymerization reaction of the first compound (and the second compound) can sufficiently proceed.

A curing temperature in Process (c) is preferably 20° C. or higher. Further, by increasing the temperature, the polymerization reaction of the first compound (and the second compound) can proceed in a short time. The curing temperature is more preferably 23° C. or higher, 40° C. or higher, 60° C. or higher, 80° C. or higher, or 100° C. or higher, and still more preferably, 150° C. or higher. By setting the curing temperature to 150° C. or higher, the polymerization reaction can proceed in a shorter time and a cyclic polyether structure can be produced. A humidity at the time of curing in Process (c) is not particularly limited, but in order to proceed hydrolysis, it is preferable to proceed the hydrolysis in an environment with moisture such as in the atmosphere (that is, more than 0% RH).

As long as the object of the exemplary embodiment of the present disclosure is achieved, the method of manufacturing a bioelectrode according to the exemplary embodiment of the present disclosure may have other processes.

### EXAMPLES

Hereinafter, the exemplary embodiment of the present disclosure will be described in more detail with reference to examples. The exemplary embodiment of the present disclosure can be implemented with appropriate modifications to the extent that they can meet the gist described above and below without limiting by the following examples, and all of them are included in the technical scope of the exemplary embodiment of the present disclosure.

### Example 1

### (a) Solution Preparing Process

53.5 parts by mass of γ-glycidoxypropylmethyldimethoxysilane (KBM402, produced by Shin-Etsu Chemical Co., Ltd.) was prepared as a liquid of a first compound having an epoxy group and an alkoxysilyl group. 2.25 parts by mass of lithium perchlorate (produced by KANTO CHEMICAL CO., INC., Cica first grade) as an alkali metal salt was added to γ-glycidoxypropylmethyldimethoxysilane, and the mixture thereof was stirred about 10 minutes while heating at 60° C. with a hot magnetic stirrer, thereby preparing a solution.

### (b) Applying Process

A borosilicate glass fiber (Pyrex (registered trademark) processed product, outer diameter: 100 μm, length: 60 cm) as an inorganic base material was immersed in the solution to apply the solution to the inorganic base material.

### (c) Curing Process

By holding the inorganic base material applied with the solution for 720 hours in an environment at 23° C. and 60% RH, the solution was cured, thereby manufacturing a bioelectrode including the inorganic base material and a conductive layer covering the inorganic base material.

In order to analyze the solution of Example 1 and a structure of the conductive layer, a total reflection FTIR spectrum was measured by a spectrometer (Shimadzu Corporation, IRPrestige-21). For the conductive layer, the solution was applied to a separate glass plate made of borosilicate glass (Pyrex (registered trademark) processed product) to manufacture a bioelectrode obtained by curing the solution under the same curing conditions, and a total reflection FTIR spectrum of a surface of the bioelectrode, that is, the conductive layer was measured.

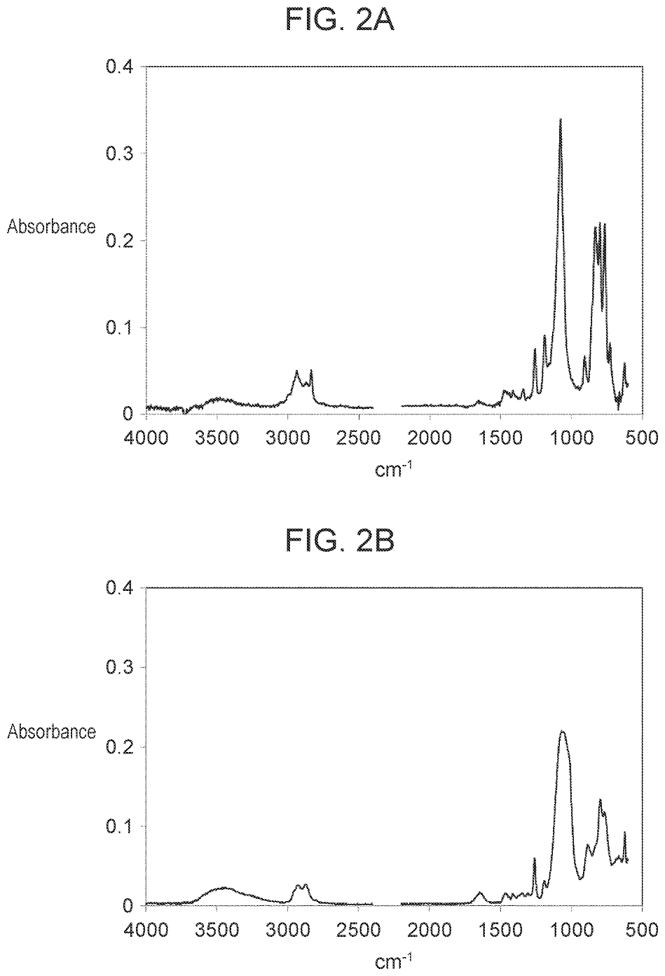

FIG. 2A shows a total reflection FTIR spectrum of the solution in Example 1, and FIG. 2B shows a total reflection FTIR spectrum of the conductive layer in Example 1. It is observed in FIG. 2A that a glycidyl ether group has a characteristic peak of 908.5 cm⁻¹ and a methoxy group has a characteristic peak of 2835.4 cm⁻¹, whereas those peaks are not observed in FIG. 2B. From this reason, it is found that ring-opening reaction of the glycidyl ether group and hydrolysis of at least the methoxy group are completed in the conductive layer after being held for 720 hours at 23° C. and 60% RH, and it is considered that the structure as illustrated in FIG. 1 is formed.

In order to examine a content of the cyclic polyether structure in the conductive layer of Example 1, an ultraviolet visible absorption spectrum was measured by a spectrophotometer (Hitachi, U-4000). A measurement sample in which the solution of Example 1 was transferred into a quartz cell having an optical path length of 5 mm and held for 720 hours at 23° C. and 60% RH, was used.

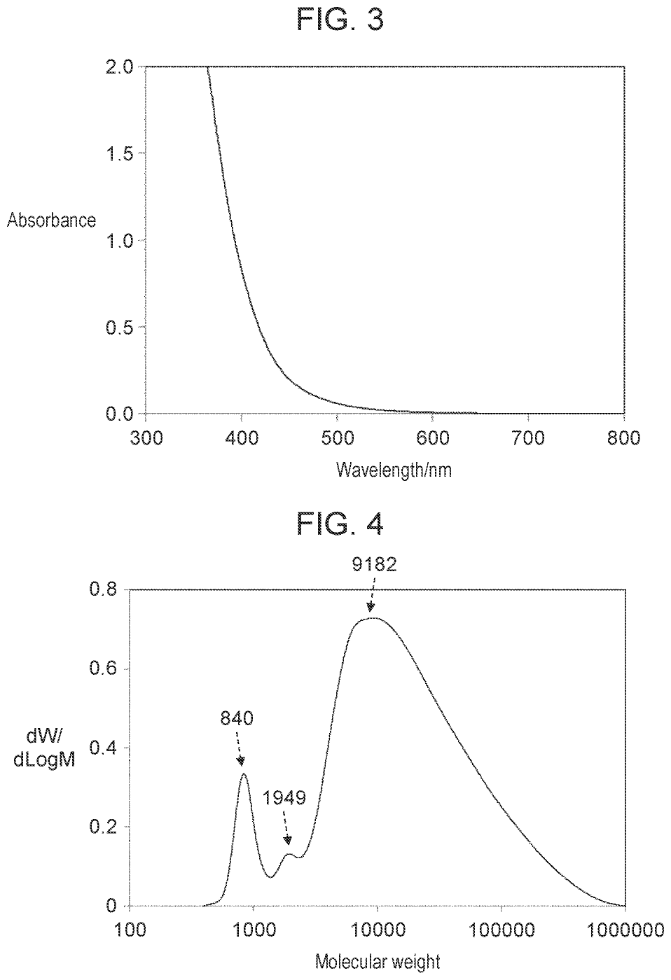

FIG. 3 shows a measurement result of the absorption spectrum of the conductive layer in Example 1. An absorbance at 450 nm was 0.199, which showed that the cyclic polyether structure was relatively small.

### Example 2

A bioelectrode was manufactured as in Example 1 except for changing Process (a) of Example 1 as follows.

37.5 parts by mass of γ-glycidoxypropylmethyldimethoxysilane (KBM402, produced by Shin-Etsu Chemical Co., Ltd.) was prepared as a first compound, 7.90 parts by mass of dimethyldimethoxysilane (KBM22, produced by Shin-Etsu Chemical Co., Ltd.) as a liquid of a second compound having a hydrocarbon group and alkoxysilyl was mixed to the first compound, 2.25 parts by mass of lithium perchlorate was added to the mixture, and the mixture was stirred with a hot magnetic stirrer for about 10 minutes while heating at 60° C., thereby preparing a solution.

### Example 3

A bioelectrode was manufactured as in Example 1 except for changing the alkali metal salt to potassium iodide (3.51 parts by mass, produced by KANTO CHEMICAL CO., INC.) in Process (a) of Example 1, changing the inorganic base material to a plate glass (Pyrex (registered trademark) processed product, area: 50 mm×50 mm, thickness: 5 mm) produced by BK7 in Process (b), and applying the solution to the inorganic base material at 500 rpm and for 10 seconds with a spin coater at the time of applying the solution.

### Example 4

A bioelectrode was manufactured as in Example 1 except for changing the inorganic base material to a columnar member made of quartz (outer diameter: 5 mm, length: 10 mm) in Process (a) of Example 1.

### Example 5

A bioelectrode was manufactured as in Example 1 except for changing the curing conditions to hold the solution for 1.5 hours at 150° C. under the atmosphere in Process (c) of Example 1. As details of the holding conditions, a borosilicate glass fiber immersed in and applied with the solution was held for 1.5 hours on a metal wire hung in a constant temperature bath of 150° C., while being fixed with a clip.

GPC measurement was carried out in order to analyze the structure of the conductive layer of Example 5 after being held for 1.5 hours at 150° C. under the atmosphere. A measurement sample in which the solution of Example 5 was transferred into a beaker and held for 1.5 hours at 150° C. in the constant temperature bath used in Example 5 was used. As a pretreatment for the GPC measurement, 5 ml of THF (containing 0.02% monoethanolamine) as a solvent was added to 100 mg of the measurement sample, the mixture thereof was stirred at about 90° C. for 2 hours, and then filtered using a filter of 0.45 μm to remove metal ion from the mixture. After the pretreatment, the GPC measurement was carried out with a GPC multi-angle light scattering photometer.

FIG. 4 shows the GPC measurement result in Example 5. In FIG. 4, a molecular weight peak was observed around 840. It is considered to form a polymer having the cyclic polyether structure represented by the following Chemical Formula 1.

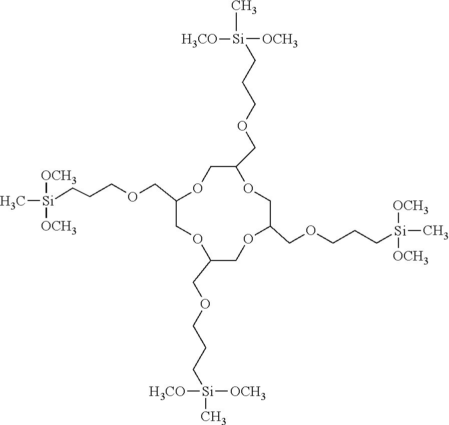

A compound of Chemical Formula 1 is obtained by cyclically polymerizing four glycidyl ether groups of γ-glycidoxypropylmethyldimethoxysilane.

Unlike the sample for GPC measurement, it is considered in the actual conductive layer that a structure in which lithium ions are coordinated as represented by the following Chemical Formula 2 is shown. However, as a result of falling off of the lithium ions in the pretreatment for GPC measurement, it is considered that the compound of Chemical Formula 1 was detected in the GPC measurement sample.

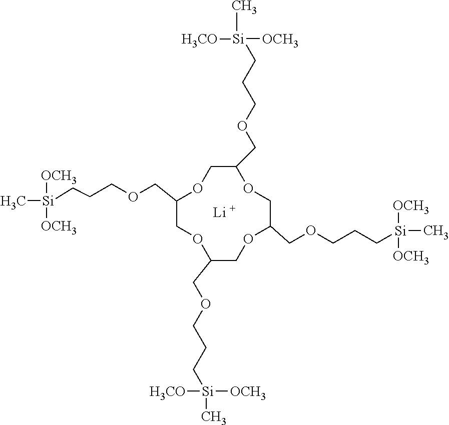

In more details, a molecular weight of the compound of Chemical Formula 1 is 880, which is larger than that in the GPC measurement result (840). Therefore, more accurately, it is attributed that the compound detected by GPC measurement is has a structure as represented by the following Chemical Formula 3.

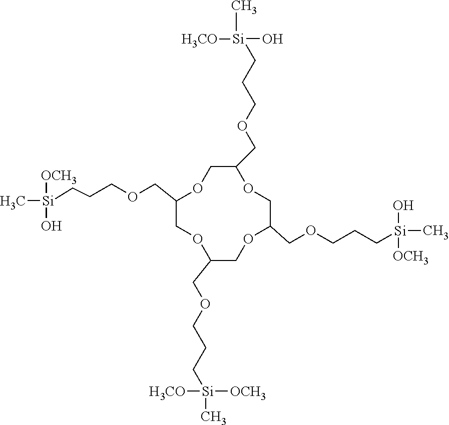

A difference between Chemical Formulas 1 and 3 is that three of eight methoxy groups are hydrolyzed to form a hydroxyl group. That is, it could be seen that even in the polymer having a cyclic polyether structure, the moiety derived from the alkoxysilyl group is hydrolyzed, which is preferable for bonding with the inorganic base material.

As the GPC measurement result, the peak of the molecular weight has a distribution around 840 (that is, broad distribution), and thus, it is considered that a mixture is produced, the mixture obtained by cyclically polymerizing the glycidyl ether group of γ-glycidoxypropylmethyldimethoxysilane with three to five or more glycidyl ether groups in the GPC measurement sample, and hydrolyzing three or more or three or less methoxy groups to a hydroxyl group in the compounds having the cyclic polyether structures.

In FIG. 4, a molecular weight peak was also observed around 1949. This peak is attributed to a peak obtained by cyclically polymerizing about nine glycidyl ether groups of γ-glycidoxypropylmethyldimethoxysilane, a peak obtained by bonding the three to five cyclically polymerized glycidyl ether groups of γ-glycidoxypropylmethyldimethoxysilane by hydrolyzing and condensing a silanol group, and the like. Each of the peaks can be also attributed to improvement of ion conductivity in terms of stable support of ions.

In FIG. 4, a molecular weight peak was also observed around 9182. The peak is attributed to that obtained by cyclically polymerizing about 40 glycidyl ether groups of γ-glycidoxypropylmethyldimethoxysilane.

In order to examine a content of the cyclic polyether of the conductive layer in Example 5 after being held for 1.5 hours at 150° C. under the atmosphere, an ultraviolet visible absorption spectrum was measured by a spectrophotometer (Hitachi, U-4000). A measurement sample in which the solution of Example 5 was transferred into a quartz cell having an optical path length of 5 mm and held for 1.5 hours at 150° C. in the constant temperature bath used in Example 5 was used.

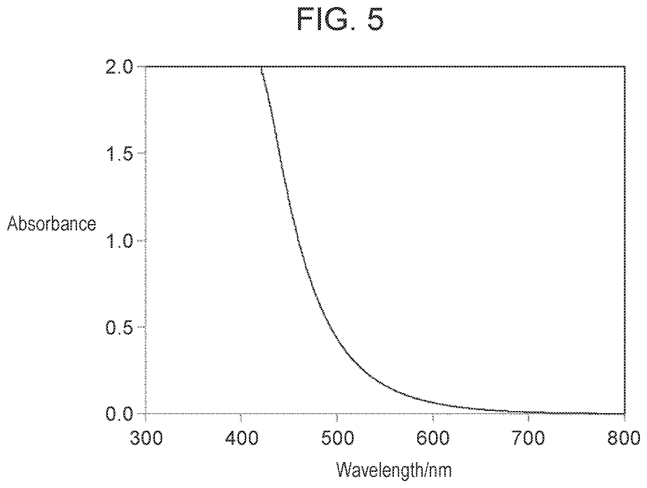

FIG. 5 shows a measurement result of the absorption spectrum of the conductive layer in Example 5. The absorbance at 450 nm was 1.23, and it was found that more cyclic polyether structures were formed as compared with Example 1 (FIG. 3).

### Example 6

A bioelectrode was manufactured as in Example 1 except for changing the first compound to 2-(3,4-epoxycyclohexyl)trimethoxysilane in Process (a) of Example 1.

### Example 7

A bioelectrode was manufactured as in Example 1 except for changing Process (a) of Example 1 as follows.

5.35 parts by mass of γ-glycidoxypropylmethyldimethoxysilane (KBM402, produced by Shin-Etsu Chemical Co., Ltd.) was prepared as a liquid of a first compound, 0.23 parts by mass of lithium perchlorate (produced by KANTO CHEMICAL CO., INC., Cica first grade) as an alkali metal salt was added to the prepared γ-glycidoxypropylmethyldimethoxysilane, and the mixture thereof was stirred about 10 minutes while heating at 60° C. with a hot magnetic stirrer. 31.4 parts by mass of copper nanoparticles (average particle diameter: 300 nm) was added to the solution and shaken manually to mix the copper nanoparticles, thereby preparing a dispersion. In this case, a volume ratio of the copper nanoparticles in the conductive layer was 40%.

### Example 8

A bioelectrode was manufactured as in Example 1 except for changing the first compound to 57.4 parts by mass of γ-glycidoxypropyltriethoxysilane (KBE403, produced by Shin-Etsu Chemical Co., Ltd.) in Process (a) of Example 1.

### Example 9

A bioelectrode was manufactured as in Example 1 except for changing the curing time to 0.5 hours in Process (c) of Example 5. Moreover, as a result of measuring the ultraviolet visible absorption spectrum in the same manner as in Example 5, the absorbance at 450 nm was 0.250.

### Example 10

A bioelectrode was manufactured as in Example 1 except for changing the curing time to 720 hours in Process (c) of Example 5. Moreover, as a result of measuring the ultraviolet visible absorption spectrum in the same manner as in Example 5, the absorbance at 450 nm was 2.50.

### Example 11

A bioelectrode was manufactured as in Example 1 except for changing the inorganic base material to a copper wire (outer diameter: 500 μm, length: 30 cm) in Process (b) of Example 1.

### Example 12

A bioelectrode was manufactured as in Example 4 except for changing the columnar member made of quartz, which is an inorganic base material, to a member having a conical end (that is, a member having a pointed structure portion) in Process (b) of Example 4. A diameter of a bottom surface of the conical portion was 5 mm, and lateral lines of the conical portion were 5 mm.

### Example 13

A bioelectrode was manufactured as in Example 2 except for changing the second compound to 12.4 parts by mass of cyclohexylmethyldimethoxysilane in Process (a) of Example 2.

### Comparative Example 1

A bioelectrode was manufactured as in Example 1 except for changing Processes (a) and (c) of Example 1 as follows.

In Comparative Example 1, 2.25 parts by mass of lithium perchlorate was added to 53.5 parts by mass of a monomer liquid of silicone rubber (addition reaction-type RTV silicone rubber, produced by Shin-Etsu Chemical Co., Ltd.), the silicone rubber being formed by a hydrosilylation reaction of vinyl group-containing organopolysiloxane by a platinum catalyst, the mixture thereof was stirred about 10 minutes while heating at 60° C. with a hot magnetic stirrer, thereby preparing a solution. Further, the solution was held for 2 hours at 150° C. after being immersed and applied, thereby manufacturing a bioelectrode.

### Comparative Example 2

Lithium perchlorate was not added in Process (a) of Example 1. A bioelectrode was manufactured in the same manner as in Example 1 except for those described above.

### Comparative Example 3

A bioelectrode was manufactured as in Comparative Example 1 except for changing the inorganic base material to that of Example 12 in Process (b) of Comparative Example 1.

Ion conductivity and adhesion of the bioelectrode obtained in each Example and each Comparative Example were evaluated.

### Measurement of Ion Conductivity

Ion conductivity was measured by filling a polytetrafluoroethylene mold having a diameter of 9.5 cm and a depth of 500 μm with the solution (or dispersion) of each Example and each Comparative Example, and curing the solution (or dispersion) under the same curing conditions as the curing process of each Example and each Comparative Example. Thereafter, the cured solution (or dispersion) was removed from the mold and a polymer was inserted into a nickel plate, thereby constituting a Swagelok cell and obtaining a measurement sample. The measurement was carried out at room temperature in a frequency range of 1 kHz to 1000 kHz.

The ion conductivity of 1.0×10⁻⁴ S/cm or more was defined as A (excellent).

The ion conductivity of 1.0×10⁻⁵ S/cm or more and less than 1.0×10⁻⁴ S/cm was defined as B (good).

The ion conductivity of less than 1.0×10⁻⁵ S/cm was defined as C (poor).

### Adhesion

When the inorganic base material was a glass plate or a columnar base material, the adhesion was evaluated by pressing an edge portion of a polyethylene plate having a thickness of 1 mm against each of the applied surface and the flat surface of the bioelectrode and gently rubbing. When the inorganic base material was a glass fiber, the adhesion was evaluated by placing the bioelectrode on a slide glass and gently rubbing it on the edge portion of the polyethylene plate. When the inorganic base material had a conical portion, the adhesion was evaluated by gently rubbing a pointed portion of the cone on the edge portion of the polyethylene plate.

When adsorption residues were observed on the inorganic base material even in a case where the conductive layer was broken without peeling off, it was evaluated as A (excellent), and when the conductive layer was peeled off and adsorption residues were not observed on the inorganic base material, it was evaluated as C (poor).

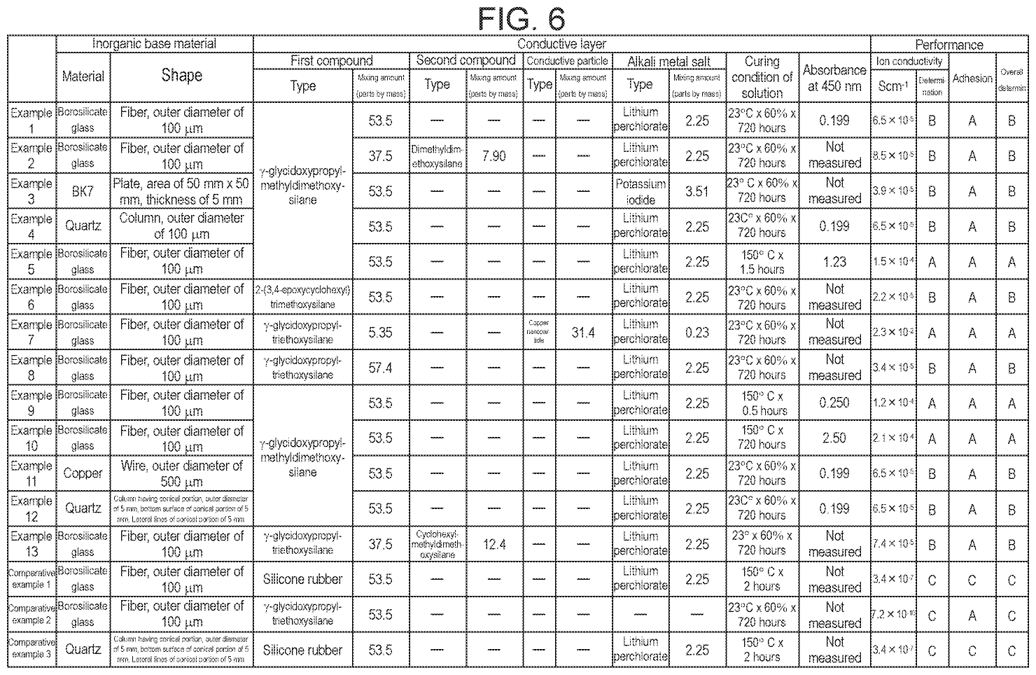

The above results are shown in FIG. 6. In the table of FIG. 6, since the conductive layers of Examples 4, 11, and 12 were the same as the conductive layer of Example 1, the measured value of Example 1 was listed in the column of ion conductivity. Since the conductive layer of Comparative Example 3 was the same as that of Comparative Example 1, the measured value of Comparative Example 1 was listed in the column of ion conductivity.

Regarding the overall determination, when all the ion conductivity and the adhesion were evaluated as A, it was determined as A (excellent), when the ion conductivity and the adhesion were evaluated as A and B in a mixed manner, it was determined as B (good), and when the ion conductivity and adhesion were evaluated as at least one C in a mixed manner, it was determined as C (poor).

From the results of FIG. 6, it can be considered as follows. Examples 1 to 13 are examples that satisfy all of the requirements specified in the exemplary embodiment of the present disclosure, and are excellent in ion conductivity and adhesion. In particular, Examples 5, 9, and 10 were different from Examples 1 to 4, 6, 8, and 11 to 13 in that the curing process was in a preferable range (curing temperature: 150° C. or higher) and the absorbance at 450 nm caused by the cyclic polyether structure was in a preferable range of 0.200 to 2.50. Therefore, the ion conductivity was excellent, and the overall determination was A. Further, unlike Examples 1 to 4, 6, 8, and 11 to 13, Example 7 had an excellent ion conductivity due to containing conductive particles, and therefore, the overall determination was A.

On the other hand, Comparative Examples 1 to 3 were examples that did not satisfy all of the requirements specified in the exemplary embodiment of the present disclosure, and therefore, the ion conductivity or the adhesion was poor.

Since Comparative Examples 1 and 3 did not contain the polymer containing the moieties derived from the first compound having an epoxy group and an alkoxysilyl group, the ion conductivity and adhesion were poor.

Since Comparative Example 2 did not contain the alkali metal ion and Group 2 element ion, the ion conductivity was poor.

From the comparison between Examples 1 and 2, it was found that the ion conductivity was improved by containing the second compound. This is because the second compound was contained, and the cross-link density of the polymer in the conductive layer was thus reduced, and the ions were easily conducted.

Further, from the comparison between Examples 2 and 13, it was found that the ion conductivity was improved when the second compound had a hydrocarbon group having 2 to 6 carbon atoms. This is because the number of carbon atoms was 2 to 6, and the cross-link density of the polymer in the conductive layer was thus reduced, and the ions were easily conducted.

The bioelectrode according to the exemplary embodiment of the present disclosure has rigidity and whose surface is mainly made of a non-metallic material to come into contact with a living body, and has high ion conductivity and excellent adhesion. Therefore, it is useful as a bioelectrode capable of obtaining a signal from a deep part of a living body or a fine part such as a nerve cell, and has a high utilization value in industry.

What is claimed is:

1. A bioelectrode comprising:
an inorganic base material; and
a conductive layer covering the inorganic base material,
wherein the conductive layer has
a polymer having moieties derived from a first compound having an epoxy group and an alkoxysilyl group, and
at least one of an alkali metal ion and a Group 2 element ion supported in the polymer, and
in the polymer, the moiety derived from the epoxy group is ring-opening polymerized, and the moiety derived from the alkoxysilyl group forms a siloxane bond.

2. The bioelectrode of claim 1, wherein the epoxy group constitutes a glycidyl ether group.

3. The bioelectrode of claim 1, wherein the siloxane bond is formed by the moiety derived from the alkoxysilyl group of the first compound, and a moiety derived from an alkoxysilyl group of a second compound having a hydrocarbon group and the alkoxysilyl group.

4. The bioelectrode of claim 1, wherein the inorganic base material is in a fibrous form having a circle equivalent diameter of 100 μm or more and 5 mm or less.

5. The bioelectrode of claim 4, wherein the inorganic base material has a pointed structure portion.

6. The bioelectrode of claim 4, wherein the inorganic base material includes glass or a metal.

7. The bioelectrode of claim 1, wherein the polymer further has a cyclic polyether structure.

8. The bioelectrode of claim 1, wherein the conductive layer further includes conductive particles.

9. The bioelectrode of claim 8, wherein the conductive particles include at least one or more selected from the group consisting of carbon, silver, and copper, and have an average particle diameter of 0.5 nm or more and 100 μm or less.

10. The bioelectrode of claim 8, wherein the conductive particles are at least one of carbon nanotubes and graphite powders.

11. A method of manufacturing a bioelectrode, the method comprising:
preparing a solution in which at least one of an alkali metal salt and a Group 2 element salt is dissolved in a liquid including a first compound having an epoxy group and an alkoxysilyl group;
applying the solution to an inorganic base material; and
curing the applied solution.

12. The method of claim 11, further comprising mixing conductive particles with the solution after the preparing of the solution and before the applying of the solution to the inorganic base material.

13. The method of claim 11, wherein the liquid further includes a second compound having a hydrocarbon group and an alkoxysilyl group.
